(19) United States
(12) Patent Application Publication
(10) Pub. No.: US 2021/0199652 A1
(43) Pub. Date: Jul. 1, 2021

(54) Test Kit For Detecting A Plurality Of Analytes

(71) Applicant: Panion & BF Biotech Inc., Taipei (TW)

(72) Inventors: Chun-Liang Shih, New Taipei (TW); Chia-Hui Liu, New Taipei (TW)

(73) Assignee: Panion & BF Biotech Inc., Taipei (TW)

(21) Appl. No.: 17/140,383

(22) Filed: Jan. 4, 2021

Related U.S. Application Data
(60) Provisional application No. 62/955,452, filed on Dec. 31, 2019.

(30) Foreign Application Priority Data
Aug. 12, 2020 (CN) 202010807460.0

(51) Int. Cl. G01N 33/543; G01N 33/569

(52) U.S. Cl. CPC G01N 33/54393; G01N 33/56983; G01N 2333/135; G01N 2333/11; G01N 2333/075

(57) ABSTRACT

The present disclosure provides a test kit for simultaneously detecting a plurality of analytes, in which the test kit includes a lysis solution and a test strip. The lysis solution includes a salt, a surfactant, a stabilizer, and a buffer solution. The test strip includes a sample pad, a conjugation pad, a cellulose membrane, and a water-absorbing pad sequentially arranged on a support plate. The conjugation pad includes a conjugation pad solution and a plurality of antibody-conjugated microspheres, and the antibody-conjugated microspheres recognize plurality of analytes. The test kit of the present disclosure achieves an effect of simultaneously detecting more than or equal to four analytes even though the conjugation pad has a limited capacity of the antibody by preparing the lysis solution with appropriate ingredients and improving a formulation of the solution contained in the test strip.

### CROSS-REFERENCE TO RELATED APPLICATION

This application claims priority to U.S. Provisional Application Ser. No. 62/955,452, filed on Dec. 31, 2019, and China Application Serial Number 202010807460.0, filed on Aug. 12, 2020, the disclosures of which are herein incorporated by reference.

### BACKGROUND

### Field of Invention

The present disclosure relates to a test kit and a method thereof, and in particular, to a test kit and a method that can simultaneously detect a plurality of analytes.

### Description of Related Art

In clinical practice, many diseases are caused by different pathogenic bacteria or viruses, but they have similar clinical symptoms (e.g., respiratory diseases caused by respiratory viruses), which poses a high risk of doctors' misjudgment of medication. Medical institutions have gradually introduced procedures for rapid pathogen screening using test kits (e.g., immunochromatography kits) before treatment, in order to prescribe the right medicine to improve the treatment effect. However, most of the current test kits can only test a single analyte in one specimen. If there is a need for simultaneous detection of a plurality of analytes, it is often necessary to collect specimens repeatedly, which will cause discomfort to the patient and affect willingness of the patient to test. Therefore, most medical institutions often only perform rapid screening of a single analyte, but cannot know whether there are still other pathogenic bacteria or viruses lurking in the body, which affects effectiveness of treatment.

The immunochromatography kit is a rapid diagnostic technique that has emerged in recent years, and is accurate, fast, and easy to operate. The principle is to pre-process the specimen with a lysis solution to improve binding of the analyte or its specific antigen. The test strip has an absorption region, a recognition region, and a indicator region in sequence. The concept of detection is as follows. The absorption region in the test strip can absorb the analyte or its specific antigen in the specimen, and the absorbed analyte or its specific antigen will gradually move in the test strip with capillary action. The analyte or its specific antigen will then bind to a specific antibody with a color molecule (e.g., a microsphere having a chromophore) in the recognition region. Next, when the analyte or its specific antigen moves to the indicator region, another specific antibody fixed on the indicator region can recognize the analyte or its specific antigen, and then display a color band in the indicator region. In this way, it can be distinguished whether there is the analyte in the specimen.

However, the test strip of the immunochromatography kit has an upper limit on maximum liquid capacity. Therefore, if there is a need for simultaneous detection of a plurality of analytes on one test strip, it is necessary to configure multiple specific antibodies, but each specific antibody will inevitably has a lower concentration compared with a single antibody used to recognize a single analyte. When there are more kinds of analytes to be detected, the upper limit of the loadable concentration of each antibody in the test strip will be lower, resulting in weaker signal and difficult to interpret.

Therefore, it is necessary to improve the current test kits and methods and propose a test kit and a method that can simultaneously detect a plurality of analytes.

### SUMMARY

In some embodiments of the present disclosure, a lysis solution is provided. The lysis solution is used for pretreatment of a rapid screening test. A weight percentage of the lysis solution is calculated as 100%. The lysis solution includes a salt, a surfactant, a stabilizer, and a buffer solution. A weight percentage of the salt is from about 0.5% to about 5%. A weight percentage of the surfactant is from about 0.5% to about 5%. A weight percentage of the stabilizer is from about 0.5% to about 5%.

In some embodiments, the weight percentage of the salt is from about 1% to about 2%, and the weight percentage of the surfactant is from about 1% to about 3%, and the weight percentage of the stabilizer is from about 1% to about 3%.

In some embodiments, the lysis solution isolates an antigen of a respiratory virus.

In some embodiments, the respiratory virus include adenovirus, influenza A (Flu A) virus, influenza B (Flu B) virus, influenza C (Flu C) virus, respiratory syncytial virus (RSV), rhinovirus (RhV), or coronavirus (CoV).

In some embodiments, the salt includes sodium chloride, potassium chloride or a combination thereof.

In some embodiments, the surfactant includes sodium dodecyl sulfate (SDS), Tween-related surfactant, Triton-related surfactant, nonylphenol (NP)-related surfactant or a combination thereof.

In some embodiments, the stabilizer includes serum protein, casein, artificial polymer or a combination thereof.

In some embodiments, the buffer solution includes phosphoric acid, carbonic acid, citric acid, acetic acid, boric acid, tris(hydroxymethyl)aminomethane, tris(hydroxymethyl)methyl-3-aminopropanesulfonic acid, N,N-bis(2-hydroxyethyl)glycine, N-tris-(hydroxymethyl)methylaminoacetic acid, 4-(2-hydroxyethyl)piperazine-1-ethanesulfonic acid hemisodium salt, 3-(N-morpholino)ethanesulfonic acid or a combination thereof.

In some embodiments, the lysis solution includes sodium azide.

In some embodiments of the present disclosure, a method for preparing an antibody-conjugated microsphere is provided, which includes following steps: providing a microsphere; providing a rinse liquid; mixing the microsphere and the rinse liquid to obtain a first mixed liquid; mixing the first mixed liquid with 1-ethyl-3-(3-dimethylaminopropyl)carbodiimide (EDC), in which 1-ethyl-3-(3-dimethylaminopropyl)carbodiimide and the microsphere in the first mixed liquid perform an activation reaction, and a time for the activation reaction is from 16 to 24 hours; mixing the microsphere after the activation reaction with a conjugation liquid to obtain a second mixed liquid; and mixing a first antibody and the second mixed liquid, so that the first antibody and the microsphere in the second mixed liquid perform a conjugation action at room temperature to obtain the antibody-conjugated microsphere.

In some embodiments, the rinse solution includes 2-(N-morpholino)ethanesulfonic acid (MES).

In some embodiments, the microsphere is a latex particle, a gold particle, a chemiluminescent group, an enzyme group or biotin.

In some embodiments, a concentration of 1-ethyl-3-(3-dimethylaminopropyl)carbodiimide in the first mixed liquid is from 0.1M to 0.5M.

In some embodiments, the step of mixing the first mixed liquid with 1-ethyl-3-(3-dimethylaminopropyl)carbodiimide further includes mixing 1-ethyl-3-(3-dimethylaminopropyl)carbodiimide, the first mixed liquid and N-hydroxysuccinimide (NHS), or mixing 1-ethyl-3-(3-dimethylaminopropyl)carbodiimide, the first mixed liquid and sulfonated N-hydroxysuccinimide.

In some embodiments, the conjugation liquid is a buffer solution, which includes phosphoric acid, carbonic acid, citric acid, acetic acid, boric acid, tris(hydroxymethyl)aminomethane, tris(hydroxymethyl)methyl-3-aminopropanesulfonic acid, N,N-bis(2-hydroxyethyl)glycine, N-tris-(hydroxymethyl)methylaminoacetic acid, 4-(2-hydroxyethyl)piperazine-1-ethanesulfonic acid hemisodium salt, 3-(N-morpholino)ethanesulfonic acid or a combination thereof.

In some embodiments, in the step of mixing the first antibody and the second mixed liquid, a weight ratio of the first antibody to the microspheres in the second mixed liquid is in a range of from about 1:40 to about 1:120.

In some embodiments of the present disclosure, a sample pad solution in an immunochromatographic test kit is provided. A weight percentage of the sample pad solution is calculated as 100%, and the sample pad solution includes a surfactant, a stabilizer, a dispersant, and a buffer solution. A weight percentage of the surfactant is from about 0.5% to about 5%. A weight percentage of the stabilizer is from about 0.05% to about 10%. A weight percentage of the dispersant is from about 0.5% to about 5%.

In some embodiments, the weight percentage of the surfactant is from about 0.5% to about 3%. The weight percentage of the stabilizer is from about 0.1% to about 0.5%. The weight percentage of the dispersant is from about 0.5% to about 1.5%.

In some embodiments, the surfactant includes sodium dodecyl sulfate, Tween-related surfactant, Triton-related surfactant, nonylphenol-related surfactant or a combination thereof.

In some embodiments, the stabilizer includes serum protein, casein, artificial polymer or a combination thereof.

In some embodiments, the dispersant includes polyvinylpyrrolidone (PVP), polyethylene glycol, oleic acid, polyacrylic acid, hydroxypropyl cellulose or a combination thereof.

In some embodiments, the buffer solution includes phosphoric acid, carbonic acid, citric acid, acetic acid, boric acid, tris(hydroxymethyl)aminomethane, tris(hydroxymethyl)methyl-3-aminopropanesulfonic acid, N,N-bis(2-hydroxyethyl)glycine, N-tris-(hydroxymethyl)methylaminoacetic acid, 4-(2-Hydroxyethyl)piperazine-1-ethanesulfonic acid hemisodium salt, 3-(N-morpholino)ethanesulfonic acid or a combination thereof.

In some embodiments, the sample pad solution further includes sodium azide.

In some embodiments of the present disclosure, a test kit for simultaneously detecting a plurality of analytes is provided, which includes a lysis solution and a test strip. The test strip includes a sample pad, a conjugation pad, a cellulose membrane, and a water-absorbing pad sequentially arranged on a support plate. The conjugation pad includes a conjugation pad solution and a plurality of antibody-conjugated microspheres, and the antibody-conjugated microspheres recognize the plurality of analytes. The antibody-conjugated microspheres are prepared by the aforementioned method.

In some embodiments, the sample pad includes a sample pad solution. One of the antibody-conjugated microspheres recognizes one of the plurality of analytes, and a surface of any one of the antibody-conjugated microspheres has a first antibody, and any one of the antibody-conjugated microspheres recognizes a specific one of the plurality of analytes through the first antibody. When the first antibodies of one group of the antibody-conjugated microspheres recognize same analyte of the plurality of analytes, the antibody-conjugated microspheres have a same color. When the first antibodies of a different group of the antibody-conjugated microspheres recognize the different analyte of the plurality of analytes, the antibody-conjugated microspheres a different color. The cellulose membrane includes a plurality of bands, which are not overlapped with each other. Any one of the bands includes a second antibody, and the second antibody recognizes the analytes, and the second antibodies on different bands recognize the different analytes. The second antibody of each of the bands and one of the first antibodies respectively recognize different sites of the same antigen of the analyte.

In some embodiments, the plurality of analytes are respiratory viruses, including adenovirus, influenza A virus, influenza B virus, influenza C virus, respiratory syncytial virus, rhinovirus, coronavirus or a combination thereof.

In some embodiments, the lysis solution is used for pretreatment prior to a test. A weight percentage of the lysis solution is calculated as 100%. The lysis solution includes a salt, a surfactant, a stabilizer, and a buffer solution. A weight percentage of the salt is from about 0.5% to about 5%. A weight percentage of the surfactant is from about 0.5% to about 5%. A weight percentage of the stabilizer is from about 0.5% to about 5%.

In some embodiments, the salt includes sodium chloride, potassium chloride or a combination thereof.

In some embodiments, the surfactant includes sodium dodecyl sulfate, Tween-related surfactant, Triton-related surfactant, nonylphenol-related surfactant or a combination thereof.

In some embodiments, the stabilizer includes serum protein, casein, artificial polymer or a combination thereof.

In some embodiments, the buffer solution includes phosphoric acid, carbonic acid, citric acid, acetic acid, boric acid, tris(hydroxymethyl)aminomethane, tris(hydroxymethyl)methyl-3-aminopropanesulfonic acid, N,N-bis(2-hydroxyethyl)glycine, N-tris-(hydroxymethyl)methylaminoacetic acid, 4-(2-Hydroxyethyl)piperazine-1-ethanesulfonic acid hemisodium salt, 3-(N-morpholino)ethanesulfonic acid or a combination thereof.

In some embodiments, the lysis solution further includes sodium azide.

In some embodiments, a weight percentage of the sample pad solution is calculated as 100%, and the sample pad solution includes a surfactant, a stabilizer, a dispersant and a buffer solution. A weight percentage of the surfactant is from about 0.5% to about 5%. A weight percentage of the stabilizer is from about 0.05% to about 10%. A weight percentage of the dispersant is from about 0.5% to about 5%.

In some embodiments, the surfactant includes sodium dodecyl sulfate, Tween-related surfactant, Triton-related surfactant, nonylphenol-related surfactant or a combination thereof.

In some embodiments, the stabilizer includes serum protein, casein, artificial polymer or a combination thereof.

In some embodiments, the dispersant includes polyvinylpyrrolidone, polyethylene glycol, oleic acid, polyacrylic acid, hydroxypropyl cellulose or a combination thereof.

In some embodiments, the buffer solution includes phosphoric acid, carbonic acid, citric acid, acetic acid, boric acid, tris(hydroxymethyl)aminomethane, tris(hydroxymethyl)methyl-3-aminopropanesulfonic acid, N,N-bis(2-hydroxyethyl)glycine, N-tris-(hydroxymethyl)methylaminoacetic acid, 4-(2-hydroxyethyl)piperazine-1-ethanesulfonic acid hemisodium salt, 3-(N-morpholino)ethanesulfonic acid or a combination thereof.

In some embodiments, the sample pad solution further includes sodium azide.

In some embodiments, the conjugation pad solution further includes a stabilizer, a dispersant, an emulsifier, and a buffer solution.

In some embodiments, a weight percentage of the conjugation pad solution is calculated as 100%, and a weight percentage of the stabilizer is from about 0.05% to about 2%, and a weight percentage of the dispersant is from about 0.05% to about 10%, and a weight percentage of the emulsifier is from about 0.1% to about 5%.

In some embodiments, the stabilizer includes serum protein, casein, artificial polymer or a combination thereof.

In some embodiments, the dispersant includes polyvinylpyrrolidone, polyethylene glycol, oleic acid, polyacrylic acid, hydroxypropyl cellulose or a combination thereof.

In some embodiments, the emulsifier includes a non-ionic emulsifier, including polyoxyl 35 castor oil, polyoxyl 40 hydrogenerated castor oil, polysorbate 20 or a combination thereof.

In some embodiments, a total concentration of the first antibody is not higher than 600 μg/mL, and a total concentration of the second antibody is not higher than 7.2 mg/mL.

In some embodiments of the present disclosure, a method for simultaneously detecting the plurality of analytes is provided, which includes following steps. A specimen is provided. A test kit is provided, which includes a test strip and a lysis solution, and the test strip includes a sample pad, a conjugation pad, a cellulose membrane, and a water-absorbing pad sequentially arranged on a support plate. The sample pad includes a sample pad solution. The conjugation pad includes a conjugation pad solution and a plurality of antibody-conjugated microspheres, and the antibody-conjugated microspheres recognize the plurality of analytes. Any one of the antibody-conjugated microspheres has a specific color and a first antibody. The cellulose membrane includes a plurality of bands, which are not overlapped with each other. Any one of the bands includes a second antibody, the second antibody recognizes the analyte, and the second antibodies on different bands recognize the different analytes. The second antibody of each of the bands and one of the first antibodies respectively recognize different sites of the same antigen of the analyte. The specimen is added to the lysis solution. The test strip is immersed in the lysis solution containing the specimen with the sample pad facing downwards, and the conjugation pad is not in contact with the lysis solution. It is determined whether the bands of the test strip are colored, and when one or more of the bands are colored, the corresponding one or more analytes are detected.

In some embodiments, a weight percentage of the lysis solution is calculated as 100%, and the lysis solution includes a salt, a surfactant, a stabilizer, and a buffer solution. A weight percentage of the salt is from about 0.5% to about 5%. A weight percentage of the surfactant is from about 0.5% to about 5%. A weight percentage of the stabilizer is between about 0.5% and about 5%.

In some embodiments, the salt includes sodium chloride, potassium chloride or a combination thereof, and the surfactant includes nonylphenol-related surfactant, and the stabilizer includes serum protein, and the buffer solution includes a phosphate buffer solution.

In some embodiments, the lysis solution further includes sodium azide.

In some embodiments, the antibody-conjugated microsphere is prepared by following steps: providing a microsphere; providing a rinse liquid; mixing the microsphere with the rinse liquid to obtain a first mixed liquid; mixing 1-ethyl-3-(3-dimethylaminopropyl)carbodiimide with the first mixed liquid, wherein 1-ethyl-3-(3-dimethylaminopropyl)carbodiimide and the microsphere in the first mixed liquid perform an activation reaction, and a time for the activation reaction is from 16 to 24 hours; mixing the microsphere after the activation reaction with a conjugation liquid to obtain a second mixed liquid; mixing a first antibody and the second mixed liquid, so that the first antibody and the microsphere in the second mixed liquid perform a conjugation action at room temperature to obtain the antibody-conjugated microsphere.

In some embodiments, the rinse solution includes 2-(N-morpholino)ethanesulfonic acid.

In some embodiments, a concentration of 1-ethyl-3-(3-dimethylaminopropyl)carbodiimide in the first mixed liquid is from 0.1M to 0.5M.

In some embodiments, the step of mixing 1-ethyl-3-(3-dimethylaminopropyl)carbodiimide with the first mixed liquid further includes mixing 1-ethyl-3-(3-dimethylaminopropyl)carbodiimide, the first mixed liquid and N-hydroxysuccinimide, or mixing 1-ethyl-3-(3-dimethylaminopropyl)carbodiimide, the first mixed liquid and sulfonated N-hydroxysuccinimide.

In some embodiments, the conjugation liquid is a phosphate buffer solution.

In some embodiments, when the first antibodies of one group of the antibody-conjugated microspheres recognize same analyte, the antibody-conjugated microspheres have a same color, and when the first antibodies of a different group of the antibody-conjugated microspheres recognize the different analyte of the plurality of analytes, the antibody-conjugated microspheres have a different color.

In some embodiments, a weight percentage of the sample pad solution is calculated as 100%, and the sample pad solution includes a surfactant, a stabilizer, a dispersant, and a buffer solution. A weight percentage of the surfactant is from about 0.5% to about 5%. A weight percentage of the stabilizer is from about 0.05% to about 10%. A weight percentage of the dispersant is from about 0.5% to about 5%.

In some embodiments, the surfactant includes nonylphenol-40 (NP-40), the stabilizer includes casein, the dispersant includes polyvinylpyrrolidone, and the buffer solution includes tris(hydroxymethyl)aminomethane.

In some embodiments, the sample pad solution further includes sodium azide.

Based on the above, the present disclosure achieves good applicability for lysing a variety of analytes through the formulation of the lysis solution having appropriate ingredients, and improves the preparation method of the microspheres to increase conjugation efficiency between the microspheres and the antibodies. Therefore, even though the conjugation pad of the test strip has a limited capacity of the antibody, the effect of detecting more than or equal to four different analytes simultaneously can be achieved, and an detection limit for each analyte is also ideal.

### BRIEF DESCRIPTION OF THE DRAWINGS

In order to make the above and other objects, features, advantages and embodiments of the present invention more obvious and understandable, the accompanying drawings are described as follows:

FIG. 1 illustrates a schematic diagram of a test kit in some embodiments of the present disclosure.

FIG. 2 illustrates a schematic diagram of a lysis reagent kit in some embodiments of the present disclosure.

FIG. 3 illustrates a schematic diagram of a test strip in some embodiments of the present disclosure.

FIG. 4 illustrates a schematic diagram of test results in some embodiments of the present disclosure.

FIG. 5 illustrates a flow chart of simultaneous detection of the plurality of analytes in some embodiments of the present disclosure.

### DETAILED DESCRIPTION

In order that the present disclosure is described in detail and completeness, implementation aspects and specific embodiments of the present disclosure with illustrative description are presented, but those are not the only form for implementation or use of the specific embodiments of the present disclosure. The embodiments disclosed herein may be combined or substituted with each other in an advantageous manner, and other embodiments may be added to an embodiment without further description. In the following description, numerous specific details will be described in detail in order to enable the reader to fully understand the following embodiments. However, the embodiments of the present disclosure may be practiced without these specific details.

As used herein, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

Although a series of operations or steps are described below to illustrate the method disclosed herein, the order of the operations or steps is not to be construed as limiting. For example, certain operations or steps may be performed in a different order and/or concurrently with other steps. In addition, not all illustrated operations, steps, and/or features are required to implement embodiments of the present disclosure. Moreover, each of the operations or steps described herein can include a plurality of sub-steps or actions.

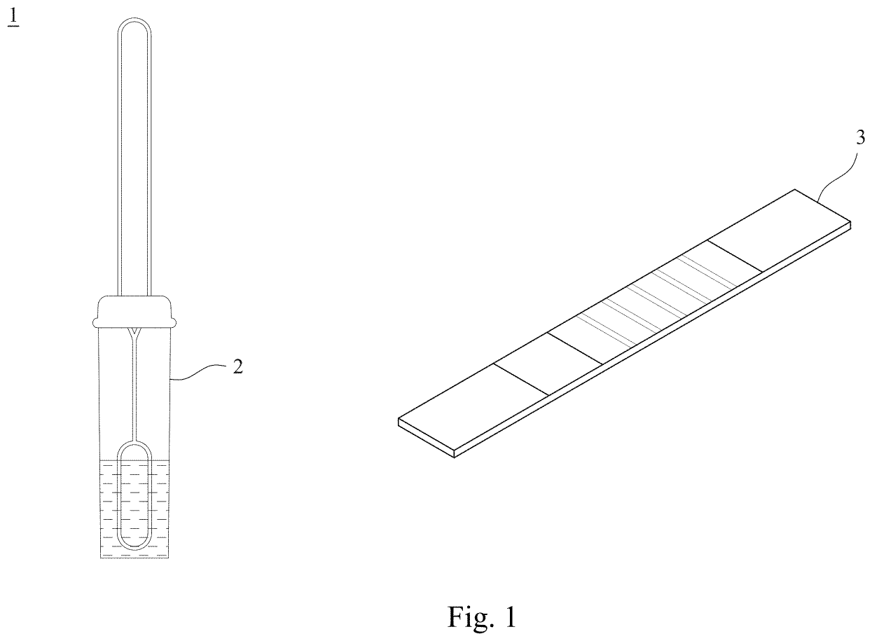

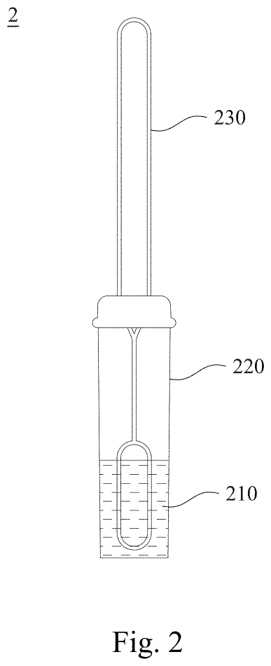

Please refer to FIG. 1 and FIG. 2. A test kit 1 for detecting the plurality of analytes simultaneously in some embodiments of the present disclosure is provided, which includes a lysis reagent kit 2 and a test strip 3. The lysis reagent kit 2 at least includes a lysis solution 210 used for a specimen pretreatment of a rapid screening test, which improves detection sensitivity of the test strip 3 to the analyte in the specimen and fluidity of the specimen. The test strip 3 is based on the principle of immunochromatography to detect whether the specimen processed by the lysis solution 210 contains antigen of the analyte to determine whether the analyte is present in the specimen.

Generally, if there is a need for simultaneous detection of the plurality of analytes, it is limited by the maximum capacity of the antibody on the test strip. Therefore, compared with the traditional test strip that only detects one analyte, there will be problems that color developing is weak, or even undetectable. The present disclosure improves the detection sensitivity of the test kit 1 to the antigen of the analyte through improvement of the lysis solution 210 and a soaking solution of the test strip 3, and thus the test kit 1 can detect the plurality of analytes (e.g., 2, 3, 4 or more kinds). The test kit 1 is helpful for real-time clinical interpretation of the results through the color developing reaction characteristics of the test strip, and can detect the plurality of analytes simultaneously, which can shorten test time and labor and material costs.

In one embodiment, the specimen is derived from the respiratory tract, such as the oral cavity, nasal cavity, trachea or bronchi. In one embodiment, the specimen may be saliva, oral mucosa, nasal mucus, nasal mucosa, tracheal secretions or/and bronchial secretions.

In one embodiment, the analyte includes a respiratory virus, such as adenovirus, influenza A virus, influenza B virus, influenza C virus, respiratory syncytial virus, rhinovirus, or coronavirus.

In some embodiments of the present disclosure, a lysis solution 210 is provided, which includes a salt, a surfactant, a stabilizer, and a buffer solution. In one embodiment, the lysis solution 210 may be suitable for isolating antigens of various respiratory viruses.

In one embodiment, the salt can make the lysis solution 210 in a hypertonic state, which can help destroy ionic bonds in the specimen's mucus and reduce viscosity of the specimen. Since the movement of the specimen on the test strip 3 is through capillary action, reducing the viscosity of the specimen can improve smoothness of the movement of the specimen on the test strip 3. If the concentration is too high, the test result of the test strip 3 will appear false positive. In some embodiments, the salt includes an alkali metal halide (e.g., potassium chloride), an alkaline earth metal halide (e.g., sodium chloride) or a combination thereof. In some embodiments, a weight percentage of the lysis solution 210 is calculated as 100%, and a weight percentage of the salt is from about 0.5% to about 5%, such as 0.5%, 1%, 1.5%, 2%, 2.5%, 3%, 3.5%, 4%, 4.5%, 5% or any value in any interval above. In one embodiment, the weight percentage of the salt is from about 1% to about 2%.

In one embodiment, the surfactant can be used to destroy the membrane-like structure (e.g., membrane protein of a virus) on the surface of the analyte to increase exposure of the antigen of the analyte and improve detectability of the antigen. In some embodiments, the surfactant includes sodium dodecyl sulfate, Tween-related surfactant (Polysorbate surfactant), Triton-related surfactant, nonylphenol-related surfactant or a combination thereof. In some embodiments, the weight percentage of the lysis solution 210 is calculated as 100%, and a weight percentage of the surfactant is from about 0.5% to about 5%, such as 0.5%, 1%, 1.5%, 2%, 2.5%, 3%, 3.5%, 4%, 4.5%, 5% or any value in any interval above. In one embodiment, the weight percentage of the surfactant is from about 1% to about 3%.

In one embodiment, the stabilizer can be used to prevent protein decomposition and non-specific recognition of the antibody to the analyte. If the concentration is too high, the test result of the test strip 3 will be false positive. In some embodiments, the stabilizer includes serum protein, casein, artificial polymer or a combination thereof. In some embodiments, the weight percentage of the lysis solution 210 is calculated as 100%, and a weight percentage of the stabilizer is from about 0.5% to about 5%, such as 0.5%, 1%, 1.5%, 2%, 2.5%, 3%, 3.5%, 4%, 4.5%, 5% or any value in any interval above. In one embodiment, the weight percentage of the stabilizer is from about 1% to about 3%.

In one embodiment, the buffer solution includes phosphoric acid, carbonic acid, citric acid, acetic acid, boric acid, tris(hydroxymethyl)aminomethane,tris(hydroxymethyl)methyl-3-aminopropanesulfonic acid, N,N-bis(2-hydroxyethyl)glycine, N-tris-(hydroxymethyl)methylaminoacetic acid, 4-(2-hydroxyethyl)piperazine-1-ethanesulfonic acid hemisodium salt, 3-(N-morpholino)ethanesulfonic acid or a combination thereof.

In an embodiment, the lysis solution 210 may include sodium azide.

In one embodiment, the lysis reagent kit 2 may optionally include a collection tube 220, a sampling rod 230, or both. The sampling rod 230 is used to collect the specimen such as the specimen derived from nasal cavity or oral mucosa. The collection tube 220 can be used to store the lysis solution 210 and acted as a reaction region for specimen pretreatment.

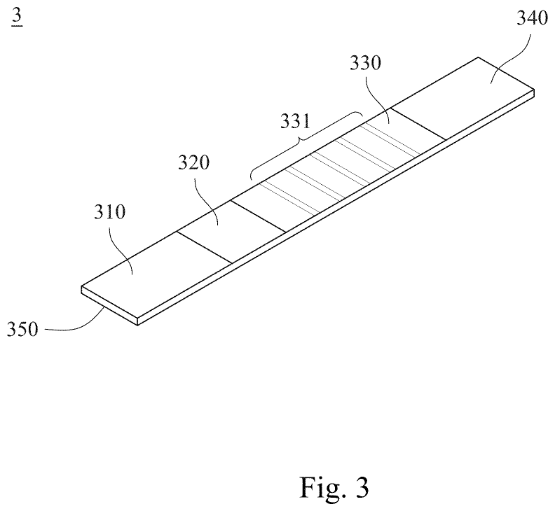

Next, referring to FIG. 3, the test strip 3 includes a sample pad 310, a conjugation pad 320, a cellulose membrane 330, and a water-absorbent pad 340 that are sequentially arranged on a support plate 350.

In one embodiment, the sample pad 310 includes a sample pad solution, which includes a surfactant, a stabilizer, a dispersant, and a buffer solution.

In one embodiment, the surfactant can be used to destroy the membrane-like structure on the surface of the analyte to increase exposure of the antigen of the analyte and improve detectability of the antigen. In some embodiments, the surfactant of the sample pad solution includes sodium dodecyl sulfate, Tween-related surfactant, Triton-related surfactant, nonylphenol-related surfactant or a combination thereof. In some embodiments, a weight percentage of the sample pad solution is calculated as 100%, and a weight percentage of the surfactant is from about 0.5% to about 5%, such as 0.5%, 1%, 1.5%, 2%, 2.5%, 3%, 3.5%, 4%, 4.5%, 5% or any value in any interval above. In one embodiment, the weight percentage of the surfactant is from about 0.5% to about 3%.

In one embodiment, the stabilizer can be used to prevent protein decomposition and non-specific recognition of the antibody to the analyte. In some embodiments, the stabilizer of the sample pad solution includes serum protein (e.g., bovine serum albumin), casein (e.g., derived from bovine milk), amino acid (e.g., glycine), artificial polymer or a combination thereof. In some embodiments, the weight percentage of the sample pad solution is calculated as 100%, and a weight percentage of the stabilizer is from about 0.05% to about 10%, such as 0.05%, 0.1%, 0.2%, 0.3%, 0.4%, 0.5%, 1%, 2%, 3%, 4%, 5%, 6%, 7%, 8%, 9%, 10% or any value in any interval above. It is worth mentioning that when the weight percentage of the stabilizer is from 0.1% to 0.5% and/or casein is used as the stabilizer, the sample pad solution can have better fluidity.

In one embodiment, the dispersant can prevent the solution from settling or agglomerating, so as to improve storage stability and maintain concentration during storage. In some embodiments, the dispersant of the sample pad solution includes polyvinylpyrrolidone (e.g., polyvinylpyrrolidone-10), polyethylene glycol, oleic acid, polyacrylic acid, hydroxypropylcellulose or a combination thereof. In some embodiments, the weight percentage of the sample pad solution is calculated as 100%, and a weight percentage of the dispersant is from about 0.5% to about 5%, such as 0.5%, 1%, 2%, 3%, 4%, 4% or any value in any interval above. In one embodiment, the weight percentage of the dispersant is from about 0.5% to about 1.5%.

In one embodiment, the buffer solution includes phosphoric acid, carbonic acid, citric acid, acetic acid, boric acid, tris(hydroxymethyl)aminomethane, tris(hydroxymethyl)methyl-3-aminopropanesulfonic acid, N,N-bis(2-hydroxyethyl)glycine, N-tris-(hydroxymethyl)methylaminoacetic acid, 4-(2-hydroxyethyl)piperazine-1-ethanesulfonic acid hemisodium salt, 3-(N-morpholino)ethanesulfonic acid or a combination thereof.

In one embodiment, the sample pad solution includes sodium azide.

In one embodiment, the conjugation pad 320 includes a conjugation pad solution and a plurality of antibody-conjugated microspheres, and the antibody-conjugated microspheres recognize a variety of analytes. In one embodiment, a surface of any one of the antibody-conjugated microspheres has a first antibody, and the antibody-conjugated microspheres can be divided into different groups according to antigen types recognized by the first antibody on the surface. Therefore, a specific group of the antibody-conjugated microspheres can recognize a specific one of the variety of analytes. In one embodiment, the first antibody detects an antigen of virus, such as respiratory virus, for example, including adenovirus, influenza A virus, influenza B virus, influenza C virus, respiratory syncytial virus, rhinovirus or coronavirus.

In one embodiment, the microspheres themselves may be directly colored (e.g., colored latex particles or gold particles), or may be a chromophore (e.g., a chemiluminescent group), or show a color after adding a suitable reaction liquid such as a suitable reaction liquid containing an enzyme group (e.g., horseradish peroxidase). In addition, other functional groups may be optionally included, such as biotin or streptavidin.

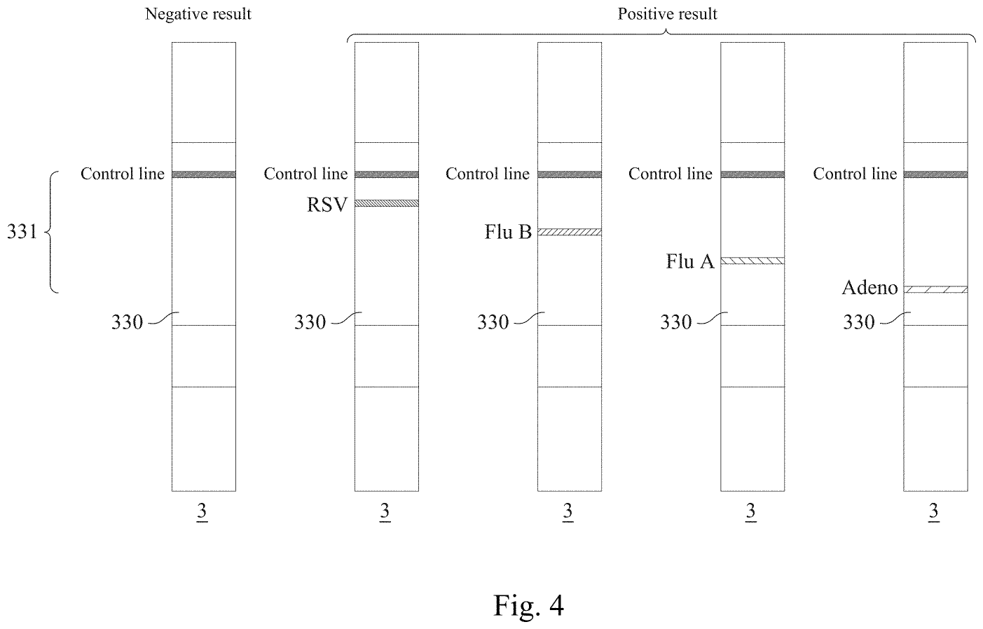

In one embodiment, referring to FIGS. 3 and 4, the cellulose membrane 330 may be made of nitrocellulose. The cellulose membrane 330 includes a plurality of bands 331, and positions of the bands 331 are not overlapped with each other, and any one of the bands 331 includes a second antibody, which can recognize the analytes. In one embodiment, the second antibody of each of the bands 331 and the specific first antibody respectively recognize different sites of the same antigen of the analyte. Therefore, the second antibodies on different bands 331 can recognize different analytes. That is, when there are the plurality of analytes in the specimen, different groups of the antibody-conjugated microspheres will respectively recognize the specific analytes. Next, after the analytes are respectively combined with the bands 331 on the cellulose membrane 330, the antibody-conjugated microspheres present its color on the cellulose membrane 330. The inspector can recognize whether there is the specific analyte according to the color developing position of the bands 331.

In one embodiment, a total concentration of the first antibody is not higher than 600 μg/mL, such as 100, 200, 300, 400, 500, 600 μg/mL, or any value in any interval above, and a total concentration of the second antibody is not higher than 7.2 mg/mL, such as 1, 2, 3, 4, 5, 6, 6.5, 7.2 mg/mL, or any value in any interval above.

In one embodiment, please also see FIG. 3 and FIG. 4. When the first antibodies on a group of the antibody-conjugated microspheres can recognize the same analyte, the group of the antibody-conjugated microspheres represents the same color simultaneously. In one embodiment, when the first antibodies of different groups of the antibody-conjugated microspheres recognize different analytes, the different groups of the antibody-conjugated microspheres represent different colors. According to this, the inspector can distinguish the type of the analytes contained in the specimen according to the color. In some embodiments, when there is respiratory syncytial virus (RSV), influenza B (Flu B) virus, influenza A (Flu A) virus, adenovirus (Adeno) or any combination of the above viruses in the specimen, the test strip 3 has the antibody-conjugated microspheres that can recognize these four viruses. For example, in the cellulose membrane 330 in FIG. 4 from top to bottom, there are a control line and color bands of RSV, Flu B, Flu A, and Adeno. The order of the bands 331 can be adjusted according to requirements and is not limited to the above embodiments. Accordingly, different bands 331 can indicate the presence or absence of the specific viruses. In addition, to simplify the interpretation, the different bands 331 are presented in different colors.

In some embodiments, a method for preparing the antibody-conjugated microspheres includes following steps: providing microspheres and providing a rinse liquid; mixing the microspheres with the rinse liquid to obtain a first mixed liquid; mixing the first mixed liquid with 1-ethyl-3-(3-dimethylaminopropyl)carbodiimide, wherein 1-ethyl-3-(3-dimethylaminopropyl)carbodiimide and the microspheres in the first mixed liquid perform an activation reaction; mixing the microspheres after the activation reaction with a conjugation liquid to obtain a second mixed liquid; and mixing the first antibody and the second mixed liquid, so that the first antibody and the microspheres in the second mixed liquid perform a conjugation action at room temperature to obtain the antibody-conjugated microspheres.

In one embodiment, the rinse liquid includes 2-(N-morpholino)ethanesulfonic acid.

In one embodiment, in the step of mixing the first mixed liquid with 1-ethyl-3-(3-dimethylaminopropyl)carbodiimide wherein 1-ethyl-3-(3-dimethylaminopropyl)carbodiimide and the microspheres in the first mixed liquid perform the activation reaction, 1-ethyl-3-(3-dimethylaminopropyl)carbodiimide in the first mixed liquid has a concentration from 0.1M to 0.5M, such as 0.1 M, 0.2M, 0.3M, 0.4M, 0.5M, or any value in any interval above.

In one embodiment, a time for the activation reaction is from 16 to 24 hours, such as 16 hours, 17 hours, 18 hours, 19 hours, 20 hours, 21 hours, 22 hours, 23 hours, 24 hours, or any value in any interval above. It is worth noting that the time for the activation reaction indicated in commercially available microspheres is generally from 15 minutes to 30 minutes. It is worth mentioning that by prolonging the time for the activation reaction of 1-ethyl-3-(3-dimethylaminopropyl)carbodiimide and the microspheres, the effect of improving a conjugation rate of the first antibody and the microspheres is achieved. As such, it is helpful to improve sensitivity of the microspheres to detect the analytes, thereby achieving the effect that the test strip 3 can be colored simultaneously to detect whether the plurality of analytes (e.g., four kinds) are present in the specimen.

In one embodiment, the step of mixing the first mixed liquid with 1-ethyl-3-(3-dimethylaminopropyl)carbodiimide further includes mixing 1-ethyl-3-(3-dimethylaminopropyl)carbodiimide, the first mixed liquid and N-hydroxysuccinimide, or mixing 1-ethyl-3-(3-dimethylaminopropyl)carbodiimide, the first mixed liquid and sulfonated N-hydroxysuccinimide. It can also be simultaneously mixed and reacted with other additives that help the activation reaction as required.

In one embodiment, the conjugation solution is a buffer solution, which includes phosphoric acid, carbonic acid, citric acid, acetic acid, boric acid, tris(hydroxymethyl)aminomethane, tris(hydroxymethyl)methyl-3-aminopropanesulfonic acid, N,N-bis(2-hydroxyethyl)glycine, N-tris-(hydroxymethyl)methylaminoacetic acid, 4-(2-hydroxyethyl)piperazine-1-ethanesulfonic acid hemisodium salt, 3-(N-morpholino)ethanesulfonic acid or a combination thereof.

In one embodiment, in the step of mixing the first antibody and the second mixed liquid, a weight ratio of the first antibody to the microspheres in the second mixed liquid is in a range of from about 1:40 to about 1:120, such as 1:40, 1:50, 1:60, 1:70, 1:80, 1:90, 1:100, 1:110, 1:120, or any ratio in any interval above. In one embodiment, after the conjugation reaction, adding ethanolamine to the solution containing the first antibody and the second mixed liquid is included. In one embodiment, after adding ethanolamine to the solution containing the first antibody and the second mixed liquid, a washing solution containing a stabilizer (e.g., casein) and a buffer solution is used to wash and suspend the microspheres.

In one embodiment, the conjugation pad solution contains a stabilizer, a dispersant, an emulsifier, and a buffer solution.

In one embodiment, the stabilizer can be used to prevent protein decomposition and non-specific recognition of the antibody to the analytes. In some embodiments, the stabilizer of the conjugation pad solution includes serum protein (e.g., bovine serum albumin), casein (e.g., derived from bovine milk), amino acid (e.g., glycine), artificial polymer or a combination thereof. In some embodiments, a weight percentage of the conjugation pad solution is calculated as 100%, and a weight percentage of the stabilizer is from about 0.05% to about 2%, such as 0.05%, 0.1%, 0.2%, 0.3%, 0.4%, 0.5%, 1%, 2% or any value in any interval above. It is worth mentioning that when the weight percentage of the stabilizer is from 0.1% to 0.5% and/or casein is used as the stabilizer, the conjugation pad solution can have better fluidity.

In one embodiment, the dispersant can prevent the solution from settling or agglomerating, so as to improve storage stability and maintain concentration during storage. In some embodiments, the dispersant of the conjugation pad solution includes polyvinylpyrrolidone (e.g., polyvinylpyrrolidone-10), polyethylene glycol, oleic acid, polyacrylic acid, hydroxypropylcellulose or a combination thereof. In some embodiments, the weight percentage of the conjugation pad solution is calculated as 100%, and a weight percentage of the dispersant is from about 0.05% to about 10%, such as 0.05%, 0.1%, 0.2%, 0.3%, 0.4%, 0.5%, 1%, 2%, 3%, 4%, 5%, 6%, 7%, 8%, 9%, 10% or any value in any interval above.

In one embodiment, the emulsifier is used to stabilize the microspheres presented on the conjugation pad 320, and the emulsifier can be a non-ionic emulsifier, which includes polyoxyl 35 castor oil, polyoxyl 40 hydrogenerated castor oil, polysorbate 20 or a combination thereof. In some embodiments, the weight percentage of the conjugation pad solution is calculated as 100%, and a weight percentage of the emulsifier is from about 0.1% to about 5%, such as 0.1%, 0.2%, 0.3%, 0.4%, 0.5%, 1%, 2%, 3%, 4%, 5% or any value in any interval above.

It is worth noting that the lysis solution 210, the sample pad solution, and the conjugation pad solution may optionally contain the same or similar ingredients, which helps to improve the smoothness of the flow of the specimen on the test strip 3.

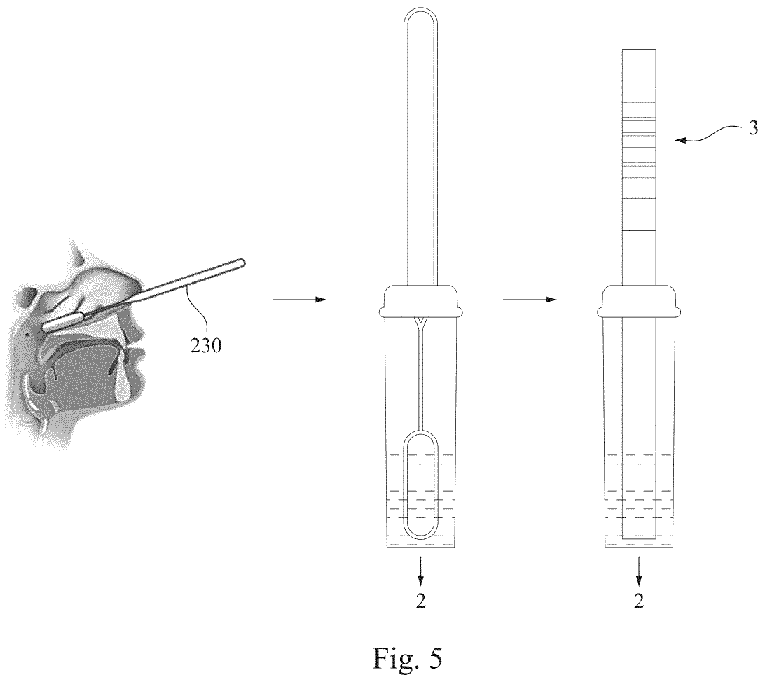

Please refer to FIGS. 2-5 simultaneously. In some embodiments of the present disclosure, a method for detecting the plurality of analytes simultaneously is provided, which includes following steps: providing a specimen; providing the test kit 1, which includes the test strip 3 and the lysis solution 210, wherein the test strip 3 includes the sample pad 310, the conjugation pad 320, the cellulose membrane 330 and the water-absorbing pad 340 sequentially arranged on the support plate 350; adding the specimen to the lysis solution 210; immersing the test strip 3 in the lysis solution 210 containing the specimen with the sample pad 310 facing downwards and the conjugation pad 320 being not in contact with the lysis solution 210 to avoid affecting sensitivity of binding between the microspheres in the solution of the conjugation pad 320 and the analytes; and determining whether the bands 331 on the test strip 3 are colored, wherein when one or more of the bands 331 are colored, it means that the specimen contains the specific analyte(s) corresponding to the colored band(s) 331.

In one embodiment, in the step of adding the specimen to the lysis solution 210, an reaction time of the specimen in the lysis solution 210 is from about 0.5 minutes to about 5 minutes, such as 0.5 minutes, 1 minute, 2 minutes, 3 minutes, 4 minutes, 5 minutes or any value in any interval above.

In one embodiment, a duration time of the step of immersing the test strip 3 in the lysis solution 210 containing the specimen with the sample pad 310 facing downwards is from about 5 minutes to about 15 minutes, such as 5 minutes, 6 minutes, 7 minutes, 8 minutes, 9 minutes, 10 minutes, 11 minutes, 12 minutes, 13 minutes, 14 minutes, 15 minutes or any value in any interval above.

In order to further test the test kit 1 provided by the various embodiments of the present disclosure and the method of simultaneously detecting the plurality of analytes, the following implementations are carried out. It should be noted that the following embodiments are provided for exemplary purposes only, rather than limiting the present invention.

### Embodiment 1. Preparation and Composition of Test Kit

First, in order to obtain a test kit that can simultaneously detect the plurality of analytes in a same specimen, in this embodiment, four respiratory viruses (i.e., adenovirus, influenza A virus, influenza B virus, and respiratory syncytial virus) were used as the analytes, and the lysis solution in the test kit and the solution in the test strip were improved.

At present, lysis solutions with different formulations were required to lyse different viruses, respectively. Therefore, if four viruses were to be lysed, four lysis solutions with different ingredients or different contents were required. This caused a certain degree of consumption of manpower and time for preparation. The lysis solution of this embodiment (see Table 1) could effectively lyse the viruses simultaneously by using one formulation, so that the four viruses could release viral antigens. In this embodiment, a solvent of the lysis solution was water. In addition, compatibility of the sample pad solution and the conjugation pad solution was also taken into consideration to improve the smoothness of the flow of the lysis solution in the test strip.

The sample pad in the test strip was soaked in the sample pad solution and then dried. The sample pad could be a glass fiber pad. The composition of the sample pad solution was listed in Table 2, and the solvent was water.

The conjugation pad in the test strip might be a polyester pad, the composition of the conjugation pad solution contained therein was listed in Table 3, and the solvent of the conjugation pad solution was water. A preparation process of the conjugation pad was as follows. First, the conjugation pad was soaked in the conjugation pad solution and then air-dried, and a latex solution was then sprayed on the conjugation pad and then dried. An amount of the latex solution could be 2 μL to 4 μL sprayed on the conjugation pad per square centimeter. The dispersant in the conjugation pad solution could promote dispersion of particles in the latex solution to avoid settling or agglomeration, so as to improve the storage stability and increase the color developing effect of the test strip.

TABLE 1
Composition of Lysis Solution

| No. | Ingredient | Content (wt %) |
|---|---|---|
| 1 | Sodium Chloride | 1.97% |
| 2 | Potassium Chloride | 0.02% |
| 3 | Sodium Phosphate Dibasic Dihydrate | 0.18% |
| 4 | Potassium Dihydrogen Phosphate | 0.03% |
| 5 | NP-40 | 1% |
| 6 | Sodium Azide | 0.08% |
| 7 | Bovine Serum Albumin (BSA) | 1% |

TABLE 2
Composition of Sample Pad Solution

| No. | Ingredient | Content (wt %) |
|---|---|---|
| 1 | Sodium Azide | 0.07% |
| 2 | Tris(hydroxymethyl)aminomethane (Tris) | 0.5% |
| 3 | Casein From Bovine Milk | 0.48% |
| 4 | Polyvinylpyrrolidone-10 (PVP-10) | 0.95% |
| 5 | NP-40 | 0.9% |
| 6 | 1N HCl Solution | 3.5% |

TABLE 3
Composition of Conjugation Pad Solution

| No. | Ingredient | Content (wt %) |
|---|---|---|
| 1 | Sodium Phosphate Dibasic Dihydrate | 0.88% |
| 2 | Casein From Bovine Milk | 0.49% |
| 3 | Polyvinylpyrrolidone-10 (PVP-10) | 0.49% |
| 4 | Sodium Azide | 0.02% |
| 5 | Polyoxyl 35 Castor Oil | 0.1% |
| | (KOLLIPHOR ® EL) | |
| 6 | 1N HCl Solution | 0.49% |

The latex solution included four antibody-conjugated microspheres that could recognize the four viruses respectively. The preparation of each of the antibody-conjugated microspheres included following steps. First, colored latex microspheres (purchased from Thermo Fisher, Catalog Number: DR1040HA, DR1040CA, DB1040HA, DB1040CA and DBK1040CA) were mixed with a rinse liquid (20 mM MES buffer, pH 6.5). The mixed liquid was ultrasonically vibrated and rinsed using the rinse liquid, and then resuspended and quantified to obtain a first mixed liquid containing 8.5 mg/mL of the latex microspheres.

Next, sulfonated N-hydroxysuccinimide was added to 1 mL of the first mixed liquid to adjust a concentration of sulfonated N-hydroxysuccinimide to 0.4M, and a vibration treatment was then performed on the first mixed liquid containing sulfonated N-hydroxysuccinimide. 1-ethyl-3-(3-dimethylaminopropyl)carbodiimide was then added to the first mixed liquid containing sulfonated N-hydroxysuccinimide to make a concentration of 1-ethyl-3-(3-dimethylaminopropyl)carbodiimide in the first mixed liquid was 0.2M. After the first mixed liquid containing 1-ethyl-3-(3-dimethylaminopropyl)carbodiimide and sulfonated N-hydroxysuccinimide was vibrated, it was rotated at room temperature to uniformly mix the first mixed liquid and to activate functional groups on the latex microspheres for a period of 16 to 24 hours.

Next, the activated latex microspheres and a conjugation solution (50 mM PB buffer, pH 7.5, Na₂HPO₄.2H₂O: 7.223 g/L, NaH₂PO₄—H₂O: 1.29807 g/L) were mixed, the latex microspheres were rinsed several times using the conjugation solution, and the latex microspheres were resuspended in the conjugation solution to obtain a second mixed liquid having a concentration of the latex microspheres from 7.5 mg/mL to 9 mg/mL.

Next, the first antibody was added to the second mixed liquid, so that a concentration (w/v) of the first antibody in the second mixed liquid was 150 μg/mL of antibody of adenovirus, 150 μg/mL of antibody of influenza A virus, 100 μg/mL of antibody of influenza B virus and 100 μg/mL of antibody of respiratory syncytial virus. After a rotation at room temperature for 2 hours, ethanolamine (working concentration was 1 M) was added, and the reaction was performed at room temperature for 30 minutes to stop the binding of the antibody and the latex microspheres.

Finally, the antibody-conjugated microspheres were rinsed using a washing solution containing a stabilizer and a buffer solution, and the microspheres were resuspended in 400 μL of the washing solution to obtain a latex liquid.

### Embodiment 2. Using Test Kit to Simultaneously Detect Four Respiratory Viruses

In order to further test the detection effect of the test kit of Embodiment 1, in the same respiratory tract specimen, detection limits of the test kit for the four respiratory viruses were determined and compared with detection limits of four commercially available kits (one respiratory virus is detected by one kit respectively). The detection limit of the virus was expressed as tissue culture infectious dose 50 (TCID₅₀). The results were listed in Table 4.

TABLE 4
Comparison of Detection Limits of Test Kit of Embodiment 1 and
Commercially Available Kit

| Test Kit of Embodiment 1 | Commercially Available Kit |
|---|---|
| Adenovirus | CerTest Adenovirus One Step Card Test |
| 2 × 10³ TCID₅₀/mL | 2 × 10³ TCID₅₀/mL |
| Flu A | Quidel ® QuickVue Influenza A/B Test |
| 2.64 × 10⁵ TCID₅₀/mL | 2.64 × 10⁵ TCID₅₀/mL |
| Flu B | Quidel ® QuickVue Influenza A/B Test |
| 3.71 × 10⁵ TCID₅₀/mL | 3.71 × 10⁵ TCID₅₀/mL |
| RSV | QuickVue RSV Test |
| 1.12 × 10³ TCID₅₀/mL | 1.12 × 10³ TCID₅₀/mL |

The results showed that the test kit provided by the embodiments of the present disclosure only need to test the specimen once to obtain the results conducted through the four commercially available test kits in the past, and the detection limits of the viruses of the test kit were equal to those of the commercially available kits.

The present disclosure achieves good applicability for lysing a variety of analytes through the formulation of the lysis solution having appropriate ingredients. In addition, the formulation of the solution contained in the test strip is improved, especially the preparation method of the microspheres, which can improve conjugation efficiency between the microspheres and the antibodies, and thus even though the conjugation pad has a limited capacity of the antibody, the effect of detecting more than or equal to four analytes simultaneously can be achieved.

The test kit of the present disclosure can detect a variety of analytes, and benefits are as follows:

1. Operation steps and time are reduced. If a commercially available kit is used, collection and detection steps need to be repeated four times, and sampling tubes and test strips need to be labeled respectively. If the number of specimens is large, the risk of wrong correspondence of the sampling tube and the test card increases, thereby increasing the waste of manpower and material resources. However, if the test kit of the present disclosure is used, the inspector only needs to perform one step, and the time of labeling and operation can be saved.

2. Probability of error is reduced. The steps have to be repeated multiple times due to the use of a variety of commercially available kits, which will increase probability of operational errors or specimen contamination. However, using the test kit of the present disclosure only needs to perform one set of steps to obtain results, which can reduce mistakes and improve accuracy.

Although the present invention has been disclosed in the above embodiments, it is not intended to limit the present invention. Anyone familiar with this technique can make various changes and modifications without departing from the spirit and scope of the present invention. The scope of protection of the invention shall be subject to the scope of appended claims.

What is claimed is:

1. A test kit for simultaneously detecting a plurality of analytes, comprising a lysis solution and a test strip, wherein the test strip comprises a sample pad, a conjugation pad, a cellulose membrane, and a water-absorbing pad sequentially arranged on a supporting plate,
wherein the conjugation pad comprises a plurality of antibody-conjugated microspheres, and the antibody-conjugated microspheres recognize a plurality of analytes.

2. The test kit of claim 1, wherein a method for preparing the antibody-conjugated microspheres comprises following steps:
providing a microsphere;
providing a rinse liquid;
mixing the microsphere with the rinse liquid to obtain a first mixed liquid;
mixing the first mixed liquid with 1-ethyl-3-(3-dimethylaminopropyl)carbodiimide; wherein 1-ethyl-3-(3-dimethylaminopropyl)carbodiimide and the microsphere in the first mixed liquid perform an activation reaction, and a time for the activation reaction is from 16 to 24 hours;
mixing the microsphere after the activation reaction with a conjugation liquid to obtain a second mixed liquid; and
mixing a first antibody and the second mixed liquid, so that the first antibody and the microsphere in the second mixed liquid perform a conjugation action at room temperature to obtain the antibody-conjugated microspheres.

3. The test kit of claim 1, wherein
one of the antibody-conjugated microspheres recognizes one of the plurality of analytes, and a surface of any one of the antibody-conjugated microspheres has a first antibody, and any one of the antibody-conjugated microspheres recognizes a specific one of the plurality of analytes through the first antibody,
wherein when the first antibodies of one group of the antibody-conjugated microspheres recognize same analyte of the plurality of analytes, the antibody-conjugated microspheres have a same color, and when the first antibodies of a different group of the antibody-conjugated microspheres recognize the different analyte of the plurality of analytes, the antibody-conjugated microspheres have a different color; and
the cellulose membrane comprises a plurality of bands, and any one of the bands comprises a second antibody, and the second antibody recognizes the plurality of analytes, and the second antibodies on different bands recognize different analytes of the plurality of analytes, wherein the second antibody of each of the bands and one of the first antibodies respectively recognize the same analyte of the plurality of analytes.

4. The test kit of claim 1, wherein the plurality of analytes are respiratory viruses, which comprises adenovirus, influenza A virus, influenza B virus, influenza C virus, respiratory syncytial virus, rhinovirus, coronavirus or a combination thereof.

5. The test kit of claim 1, wherein the lysis solution is used for pretreatment, and a weight percentage of the lysis solution is calculated as 100%, and the lysis solution comprises:
a salt with a weight percentage from about 0.5% to about 5%;
a surfactant with a weight percentage from about 0.5% to about 5%;
a stabilizer with a weight percentage from about 0.5% to about 5%; and
a buffer solution.

6. The test kit of claim 5, wherein the salt comprises sodium chloride, potassium chloride or a combination thereof.

7. The test kit of claim 5, wherein the surfactant comprises sodium dodecyl sulfate, Tween-related surfactant, Triton-related surfactant, nonylphenol-related surfactant or a combination thereof.

8. The test kit of claim 5, wherein the stabilizer comprises serum protein, casein, artificial polymer or a combination thereof.

9. The test kit of claim 5, wherein the lysis solution further comprises sodium azide.

10. The test kit of claim 1, wherein the sample pad comprises a sample pad solution, and a weight percentage of the sample pad solution is calculated as 100%, and the sample pad solution comprises:
a surfactant with a weight percentage from about 0.5% to about 5%;
a stabilizer with a weight percentage from about 0.05% to about 10%;
a dispersant with a weight percentage from about 0.5% to about 5%; and
a buffer solution.

11. The test kit of claim 10, wherein the surfactant comprises sodium dodecyl sulfate, Tween-related surfactant, Triton-related surfactant, nonylphenol-related surfactant or a combination thereof.

12. The test kit of claim 10, wherein the stabilizer comprises serum protein, casein, artificial polymer or a combination thereof.

13. The test kit of claim 10, wherein the dispersant comprises polyvinylpyrrolidone, polyethylene glycol, oleic acid, polyacrylic acid, hydroxypropylcellulose or a combination thereof.

14. The test kit of claim 10, wherein the sample pad solution further comprises sodium azide.

15. The test kit of claim 1, wherein the conjugation pad further comprises a conjugation pad solution, and the conjugation pad solution comprises a stabilizer, a dispersant, an emulsifier, and a buffer solution.

16. The test kit of claim 15, wherein a weight percentage of the conjugation pad solution is calculated as 100%, and
a weight percentage of the stabilizer is from about 0.05% to about 2%,
a weight percentage of the dispersant is from about 0.05% to about 10%,
and a weight percentage of the emulsifier is from about 0.1% to about 5%.

17. The test kit of claim 15, wherein the stabilizer comprises serum protein, casein, artificial polymer or a combination thereof.

18. The test kit of claim 15, wherein the dispersant comprises polyvinylpyrrolidone, polyethylene glycol, oleic acid, polyacrylic acid, hydroxypropyl cellulose or a combination thereof.

19. The test kit of claim 15, wherein the emulsifier comprises a non-ionic emulsifier, which comprises polyoxyl 35 castor oil, polyoxyl 40 hydrogenerated castor oil, polysorbate 20 or a combination thereof.

20. The test kit of claim 3, wherein a total concentration of the first antibody is not higher than 600 μg/mL, and a total concentration of the second antibody is not higher than 7.2 mg/mL.
